(19) United States
(12) Patent Application Publication
(10) Pub. No.: US 2026/0000793 A1
(43) Pub. Date: Jan. 1, 2026

(54) MYOCARDITIS-TARGETED RADIONUCLIDE MOLECULAR PROBE, PREPARATION METHOD THEREFOR, AND APPLICATION THEREOF

(71) Applicant: Tongji Hospital, Tongji Medical College, Huazhong University of Science and Technology, Wuhan (CN)

(72) Inventors: Xiaoquan RAO, Wuhan (CN); Dawei JIANG, Wuhan (CN); Jixin ZHONG, Wuhan (CN); Wenzhu HU, Wuhan (CN); Wanhao GAO, Wuhan (CN)

(73) Assignee: Tongji Hospital, Tongji Medical College, Huazhong University of Science and Technology, Wuhan (CN)

(21) Appl. No.: 19/202,402

(22) Filed: May 8, 2025

(30) Foreign Application Priority Data
Jun. 27, 2024 (CN) 2024108500860

(51) Int. Cl. A61K 51/04

(52) U.S. Cl. CPC A61K 51/0459; A61K 51/0497; A61K 2123/00

(57) ABSTRACT

Disclosed are a myocarditis-targeted radionuclide molecular probe, a preparation method therefor, and an application thereof. The myocarditis-targeted radionuclide molecular probe includes a DPP4 inhibitor, a bifunctional chelator covalently bonded to an amino group of the DPP4 inhibitor, and a metallic radionuclide coordinately bonded to the bifunctional chelator. The myocarditis-targeted radionuclide molecular probe provided by the present disclosure exhibits high specificity and good targeting capability, and demonstrates significant uptake in myocardial inflammatory cells, and low uptake in non-target tissues. Characterized by high resolution and sensitivity of imaging, the myocarditis-targeted radionuclide molecular probe is applied for early diagnosis of cardiac inflammatory infiltration severity, therapeutic efficacy evaluation, prognosis assessment and the like, and has promising clinical and application prospects in the field of non-invasive nuclear medicine diagnosis and treatment as a myocarditis imaging agent.

### CROSS-REFERENCE TO RELATED APPLICATIONS

The application claims priority to Chinese patent application No. 2024108500860, filed on Jun. 27, 2024, with the entire contents incorporated herein by reference.

### TECHNICAL FIELD

The present disclosure relates to the technical field of radionuclide molecular probes, and in particular to a myocarditis-targeted radionuclide molecular probe, a preparation method therefor, and an application thereof.

### BACKGROUND

Myocarditis refers to localized or diffuse inflammatory lesions of myocardium caused by nonischemic factors, and is histologically characterized by infiltration of inflammatory cells in the myocardial interstitium, and accompanied by cardiomyocyte degeneration and necrosis. Based on etiological differences, myocarditis is classified into infectious myocarditis and non-infectious myocarditis. The infectious myocarditis from viral infections is the most common. Various viruses cause or induce myocarditis, including coxsackievirus A, coxsackievirus B, adenovirus, cytomegalovirus, Japanese encephalitis virus, Epstein-Barr virus, influenza virus, parainfluenza virus, herpes simplex virus and the like. Clinical symptoms of myocarditis are atypical, mainly depending on the extent and severity of lesions. A minority of cases remain entirely asymptomatic. Mild symptoms are non-specific, including fever, cough, diarrhea and the like. Severe symptoms include severe arrhythmia, heart failure, dyspnea, or fatigue. Critical symptoms include cardiogenic shock, refractory arrhythmia, and even death. Therefore, significant challenges exist in diagnosis and treatment of myocarditis.

Conventional diagnostic methods, including medical history analysis, electrocardiography (ECG), serum biomarkers, echocardiography and cardiac magnetic resonance (CMR) imaging, exhibit insufficient sensitivity and specificity and cannot serve as reliable diagnostic criteria. Endomyocardial biopsy is required for definitive diagnosis, and an invasive method is required to obtain myocardial tissues. Due to significant risks, this method cannot be popularized and is severely limited in clinical applications. In recent years, radionuclide imaging technology has initially demonstrated its role in biological imaging, which enables real-time, on-site and non-invasive imaging. Some researchers have explored use of radioactive imaging agents for early diagnosis of myocarditis, and the radioactive imaging agents include a commonly used non-targeted probe ¹⁸F fluorodeoxyglucose or a targeted probe ¹⁸F-fluoromethyl-PBR28 developed for translocator proteins. However, applications of the above radioactive imaging agents in cardiac inflammation imaging are limited. For example, ¹⁸F fluorodeoxyglucose is a non-specific functional imaging probe taken up by cardiomyocytes in large quantities, thereby interfering with detection of cardiac inflammatory foci. The ¹⁸F-fluoromethyl-PBR28 probe exhibits inadequate sensitivity for detecting myocardial inflammation, and is more suitable for detecting extracardiac inflammation. Therefore, it is urgent to find a non-invasive or low-invasive myocarditis detection method with high specificity and sensitivity for myocardial inflammatory foci, which is of great significance for clinical diagnosis and treatment of myocarditis.

### SUMMARY

In view of deficiencies in the prior art including lack of myocarditis-targeted molecular probes for targeted, specific, highly sensitive, and/or non-invasive detection of myocardial inflammatory foci, the present disclosure provides a myocarditis-targeted radionuclide molecular probe and a preparation method therefor, and further provides an application of the myocarditis-targeted radionuclide molecular probe in preparing a myocarditis imaging agent, as well as a myocarditis imaging agent containing the foregoing myocarditis-targeted radionuclide molecular probe.

To achieve the above objective, the present disclosure is specifically achieved by means of the following technical solution:

In a first aspect, the present disclosure provides a myocarditis-targeted radionuclide molecular probe, including a DPP4 inhibitor, a bifunctional chelator covalently bonded to an amino group of the DPP4 inhibitor, and a metallic radionuclide coordinately bonded to the bifunctional chelator, where the DPP4 inhibitor is at least one of Sitagliptin, Saxagliptin, Alogliptin, Linagliptin, and Gemigliptin.

Further, the DPP4 inhibitor is Linagliptin.

Further, the bifunctional chelator is at least one of DOTA-NHS-ester, NOTA-NHS-ester, p-SCN-Bn-NOTA, p-SCN-Bn-DOTA, p-SCN-Bn-PCTA, p-SCN-Bn-HEHA, p-SCN-Bn-TCMC, and p-SCN-Bn-DTPA.

Furthermore, the bifunctional chelator is at least one of p-SCN-Bn-NOTA, p-SCN-Bn-DOTA, p-SCN-Bn-PCTA, p-SCN-Bn-HEHA, p-SCN-Bn-TCMC, and p-SCN-Bn-DTPA.

Furthermore, the bifunctional chelator is p-SCN-Bn-DOTA.

Further, the metallic radionuclide is at least one of ⁶⁸Ga, ¹⁸F-Al, ⁶⁴Cu, ⁸⁹Zr, ^{99m}Tc, ¹¹¹In, ¹⁸⁸Re, ¹⁸⁶Re, ¹⁷⁷Lu, and ⁹⁰Y.

Furthermore, the metallic radionuclide is ⁶⁸Ga.

In a second aspect, the present disclosure provides a preparation method for the above myocarditis-targeted radionuclide molecular probe, and the method includes the following steps:
S1, dissolving a DPP4 inhibitor and a bifunctional chelator in an organic solvent, reacting same under alkaline conditions, and purifying after the reaction to obtain a precursor compound; and
S2, adding a metallic radionuclide eluent and the precursor compound into a buffer solution to perform a metallic radionuclide labeling reaction, and separating and purifying after the reaction to obtain a myocarditis-targeted radionuclide molecular probe.

Further, in S1, a molar ratio of the DPP4 inhibitor to the bifunctional chelator is 1-2:1.

Further, in S1, the organic solvent is dimethyl sulfoxide, methanol or chloroform.

Further, in S1, a pH value of the reaction is 8.5-9.5, and the reaction is performed at 20-35° C. with shaking in a dark for 1-3 h or at 4° C. overnight.

Further, in S1, high-performance liquid chromatography is employed to separate and purify the precursor compound.

Further, in S2, a dosage ratio of metallic radionuclide in the metallic radionuclide eluent to the precursor compound is 30-50 MBq:1 nM.

Further, in S2, the buffer solution is a sodium acetate buffer solution with a concentration of 0.1-1 M.

Further, in S2, a pH value of the reaction is 4.0-4.5, and the reaction is performed at 90-100° C. with shaking for 10-20 min.

Further, in S2, a solid-phase extraction C18 column is used to separate and purify the myocarditis-targeted radionuclide molecular probe.

In a third aspect, the present disclosure provides an application of the above myocarditis-targeted radionuclide molecular probe in preparing a myocarditis imaging agent.

Further, the myocarditis is viral myocarditis.

In a fourth aspect, the present disclosure provides a myocarditis imaging agent, including the above myocarditis-targeted radionuclide molecular probe.

The advantages and positive effects of the present disclosure are as follows:

The myocarditis-targeted radionuclide molecular probe provided by the present disclosure exhibits high specificity and good targeting capability, and demonstrates significant uptake in myocardial inflammatory cells, and low uptake in non-target tissues. Characterized by high resolution and sensitivity of imaging, the myocarditis-targeted radionuclide molecular probe is applied for early diagnosis of cardiac inflammatory infiltration severity, therapeutic efficacy evaluation, prognosis assessment and the like, and has promising clinical potential and application prospects in the field of non-invasive nuclear medicine diagnosis and treatment as a myocarditis imaging agent.

### BRIEF DESCRIPTION OF DRAWINGS

To describe the technical solutions in the examples of the present disclosure more clearly, the accompanying drawings required for describing the examples are briefly described below. Apparently, the accompanying drawings in the following description show merely some examples of the present disclosure, and those of ordinary skill in the art may still derive other drawings from these accompanying drawings without creative efforts.

FIG. 1 is a mass spectrogram of a precursor compound of a myocarditis-targeted radionuclide molecular probe synthesized in an example of the present disclosure.

FIG. 2 is an ultraviolet spectrogram of a precursor compound of a myocarditis-targeted radionuclide molecular probe synthesized in an example of the present disclosure.

FIG. 3 shows high-performance liquid chromatograms of a myocarditis-targeted radionuclide molecular probe before and after purification according to an example of the present disclosure.

FIG. 4 illustrates pathological detection results of cardiac tissues of myocarditis model mice according to an example of the present disclosure, where FIG. 4A shows results of HE staining and immunohistochemical staining, and FIG. 4B shows results of immunofluorescence analysis.

FIG. 5 demonstrates experimental results of cellular uptake with a radionuclide molecular probe according to an example of the present disclosure, where FIG. 5A shows expression levels of DPP4 protein in 293T cells overexpressing DPP4, and FIG. 5B shows uptake rates of 293T cells highly expressing DPP4 against a radionuclide molecular probe.

FIG. 6 illustrates experimental results of in vivo targeting of a radionuclide molecular probe according to an example of the present disclosure, where FIG. 6A shows mice not subcutaneously injected at axillary regions thereof, and FIG. 6B shows mice subcutaneously injected with a suspension of 293T cells overexpressing DPP4 protein at axillary regions thereof.

FIG. 7 illustrates PET/CT imaging results of a myocarditis model mouse detected with a radionuclide molecular probe according to an example of the present disclosure, where FIG. 7A shows imaging results of the mouse on the third day before viral infection, FIG. 7B shows imaging results of the mouse on the seventh day after viral infection, and FIG. 7C is a statistical graph showing imaging results on radiation uptake rates of cardiac regions of the mouse before and after viral infection.

### DETAILED DESCRIPTION OF THE EMBODIMENTS

To make the objectives, technical solutions and advantages of the present disclosure clearer, the present disclosure is described in further detail below in conjunction with the examples. The examples described herein are merely used to explain the present disclosure, and are not intended to limit the present disclosure.

Based on the information contained in the present disclosure, those skilled in the art easily make various modifications to the precise description of the present disclosure without departing from the spirit and scope of the appended claims. It is to be understood that the scope of the present disclosure is not limited to the defined processes, properties or components, because these embodiments and other descriptions are merely illustrative of specific aspects of the present disclosure. In fact, various modifications obviously made to the embodiments of the present disclosure by those skilled in the art or related fields all fall within the scope of the appended claims.

In order to better understand the present disclosure but not to limit the scope of the present disclosure, all the numbers and other numerical values used in the present disclosure to express the amount, percentage and the like, are to be understood as being modified by the word “about” in all cases. Therefore, unless otherwise specified, the numerical parameters listed in the specification and the appended claims are approximate, which may be changed based on different desired properties sought to be achieved. Each numerical parameter is at least construed as being obtained according to reported significant figures and conventional rounding methods.

In addition, it is to be noted that, unless otherwise defined, in the context of the present disclosure, scientific and technical terms used have the meanings commonly understood by those of ordinary skill in the art. The meanings of the terms “including”, “comprising”, “containing”, “having” and other similar words and expressions are non-restrictive, that is, other steps and other components that do not materially affect the results are optionally added as needed. The term “and/or” is to be interpreted as explicit disclosure of either of two specified features or components both in the presence and absence of the other. For example, the term “A and/or B” is construed as including the following cases: (i) A, (ii) B, and (iii) A and B.

To make the above objectives, features and advantages of the present disclosure easier to understand, the present disclosure is described in detail below.

Compared with molecular probes such as ¹⁸F fluorodeoxyglucose or ¹⁸F-fluoromethyl-PBR28, radioactive imaging agents synthesized by using targeted compounds are characterized by strong tissue penetration and high specificity. However, in the current research on radionuclide imaging agents for myocarditis, no radionuclide-labeled targeted small-molecule compounds have been reported.

In the present disclosure, a myocarditis model is constructed based on data of mice infected with coxsackievirus B. Study results of viral myocarditis show that dipeptidyl peptidase-4 (DPP4) protein is specifically and highly expressed in inflammatory cell infiltration regions of myocarditis, and intrinsic cardiomyocytes and cardiac fibroblasts hardly express DPP4 protein, indicating that DPP4 is optionally used as a biological target for myocarditis imaging.

Based on the above findings, a DPP4 inhibitor is selected as a central structure in the present disclosure. The DPP4 inhibitor is selected from Sitagliptin, Saxagliptin, Alogliptin, Linagliptin, and Gemigliptin. These chemicals are small-molecule compounds with high target specificity toward DPP4. Taking Linagliptin as an example, Linagliptin is a non-peptidomimetic DPP4-targeting compound with a xanthine structure, which forms a non-covalent bond with groups of an active site of DPP4. As a small molecule drug that has been applied clinically for many years, Linagliptin is beyond doubt in pharmacological safety, with a structural formula as follows:

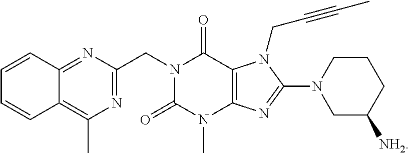 [SMILES: CC#CCn1c(N2CCC[C@@H](N)C2)nc2c1c(=O)n(Cc1nc(C)c3ccccc3n1)c(=O)n2C]

A structural formula of Sitagliptin is as follows:

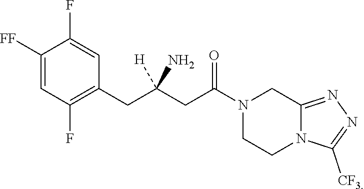

A structural formula of Saxagliptin is as follows:

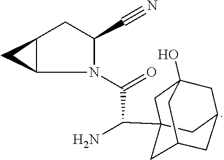 [SMILES: N#C[C@@H]1C[C@@H]2C[C@@H]2N1C(=O)[C@@H](N)C12CC3CC(CC(O)(C3)C1)C2]

A structural formula of Alogliptin is as follows:

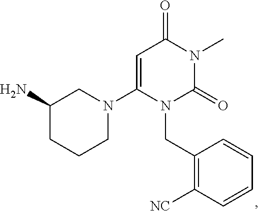 [SMILES: Cn1c(=O)cc(N2CCC[C@@H](N)C2)n(Cc2ccccc2C#N)c1=O]

A structural formula of Gemigliptin is as follows:

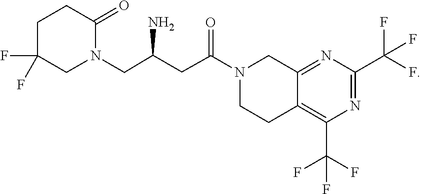 [SMILES: N[C@@H](CC(=O)N1CCc2c(nc(C(F)(F)F)nc2C(F)(F)F)C1)CN1CC(F)(F)CCC1=O]

In the present disclosure, a molecular probe is formed by simultaneously chelating a radionuclide and conjugating a molecule-targeted DPP4 inhibitor through a bifunctional chelator. The bifunctional chelator is a chelator containing two reactive groups simultaneously, where one of the reactive groups is covalently bonded to groups (e.g., —NH₂, —SH, or —OH) of biological molecules such as polypeptides and proteins, including a carboxyl group (—COOH), a succinate group (—NHS), a thiocyanate group (—NCS) and the like, and the other reactive group is a coordinating atom on a heterocyclic ring, which is combined with metallic radionuclides (such as Ga and Cu) to form a stable complex.

On this basis, the present disclosure provides a myocarditis-targeted radionuclide molecular probe in an example, including a DPP4 inhibitor, a bifunctional chelator covalently bonded to an amino group of the DPP4 inhibitor, and a metallic radionuclide coordinately bonded to the bifunctional chelator, where the DPP4 inhibitor is at least one of Sitagliptin, Saxagliptin, Alogliptin, Linagliptin, and Gemigliptin.

In the present disclosure, a bifunctional chelator reacts with a primary amino group of a DPP4 inhibitor to form a stable covalent bond, which enables to place a complete structure of the DPP4 inhibitor outside, and retains specificity and activity of binding to DPP4. Concurrently, the bifunctional chelator reacts with a metallic radionuclide to form a coordination bond and obtain a radionuclide molecular probe. The radionuclide molecular probe provided by the present disclosure exhibits good affinity and targeting capability for inflammatory cells of myocarditis highly expressing DPP4 protein. The radionuclide molecular probe of the present disclosure is injected into model mice of viral myocarditis induced by coxsackievirus B3 (CVB3) and then imaging is performed. It is found that the probe exhibits significant uptake in myocardial inflammatory foci of the myocarditis model mice and low uptake in non-target tissues, indicating strong specificity and targeting capability. Characterized by high resolution and sensitivity of imaging, the myocarditis-targeted radionuclide molecular probe is applied for early diagnosis of cardiac inflammatory infiltration severity, therapeutic efficacy evaluation, prognosis assessment and the like, and has promising clinical and application prospects in the field of non-invasive nuclear medicine diagnosis and treatment as a myocarditis imaging agent. Additionally, the DPP4 inhibitor selected in the present disclosure has been proven to have good biosafety through many years of pharmaceutical applications, and the prepared molecular probe demonstrates good biosafety with a safe metabolic pathway, without impairment to organ functions.

Preferably, the DPP4 inhibitor is Linagliptin. The bifunctional chelator applicable to the present disclosure is selected from DOTA, NOTA, NETA, PCTA, HEHA, TCMC, DTPA, hynic and derivatives thereof, specifically, including but not limited to: 1) DOTA-NHS-ester and NOTA-NHS-ester, where either of such chelators contains a succinate group (—NHS), and is an activatable esterification reagent, and when reacting with a molecule containing an amino group, NHS ester forms an amide bond with the amino group, such that DOTA and NOTA ester groups are connected to molecules of the DPP4 inhibitor; and 2) p-SCN-Bn-NOTA, p-SCN-Bn-DOTA, p-SCN-Bn-PCTA, p-SCN-Bn-HEHA, p-SCN-Bn-TCMC, and p-SCN-Bn-DTPA, where each of such chelators contains a reactive electrophilic group-thiocyanate group (—NCS), which undergoes a nucleophilic substitution reaction with an amino group of the DPP4 inhibitor under alkaline conditions (pH 8.5 to pH 9.5) to form a stable thiourea bond (—NH—C(═S)—NH—).

Preferably, the bifunctional chelator is at least one of p-SCN-Bn-NOTA, p-SCN-Bn-DOTA, p-SCN-Bn-PCTA, p-SCN-Bn-HEHA, p-SCN-Bn-TCMC, and p-SCN-Bn-DTPA.

A radioactive metallic radionuclide is used as a tracer. Based on targeted distribution of radionuclide molecular probes in myocardial inflammatory focus cells in vivo, tissue-penetrating rays emitted by radionuclides during nuclear decay are detected and recorded in vitro by an instrument, to achieve the purpose of locating and qualitatively diagnosing myocarditis. Therefore, the present disclosure does not specifically limit the types of metallic radionuclides, and any radionuclide capable of spontaneously emitting tissue-penetrating rays is allowed, where the rays include but are not limited to beta (B) rays and gamma (γ) rays.

Metallic radionuclides applicable to the present disclosure include, but are not limited to: ⁶⁸Ga, ¹⁸F-Al, ⁶⁴Cu, ⁸⁹Zr, ^{99m}Tc, ¹¹¹In, ¹^{₈₈}Re, ¹⁸⁶Re, ¹⁷⁷Lu, and ⁹⁰Y.

In a preferred embodiment, the bifunctional chelator is p-SCN-Bn-DOTA, and the metallic radionuclide is ⁶⁸Ga.

Imaging methods include, but are not limited to, positron emission tomography/X-ray computed tomography (PET/CT), positron emission tomography/magnetic resonance imaging (PET/MR), positron emission tomography (PET), single-photon emission computed tomography/X-ray computed tomography (SPECT/CT), single-photon emission computed tomography (SPECT), and magnetic resonance imaging (MRI).

In another example, the present disclosure provides a preparation method for the above myocarditis-targeted radionuclide molecular probe, and the method includes the following steps:
S1, dissolve a DPP4 inhibitor and a bifunctional chelator in an organic solvent, reacting same under alkaline conditions, and purify after the reaction to obtain a precursor compound; and
S2, add a metallic radionuclide eluent and the precursor compound into a buffer solution to perform a metallic radionuclide labeling reaction, and separate and purify after the reaction to obtain a myocarditis-targeted radionuclide molecular probe.

The preparation method for the above myocarditis-targeted radionuclide molecular probe and its advantages compared with the prior art are not described in detail herein. Additionally, the preparation method adopted in the present disclosure is simple and features a high yield, a fast reaction speed, and low requirements for reaction equipment.

Optionally, the DPP4 inhibitor is at least one of Sitagliptin, Saxagliptin, Alogliptin, Linagliptin, and Gemigliptin, and preferably Linagliptin.

Optionally, in S1, a molar ratio of the DPP4 inhibitor to the bifunctional chelator is 1-2:1.

Optionally, in S1, the organic solvent is dimethyl sulfoxide (DMSO), methanol or chloroform.

Optionally, in S1, a pH value of the reaction is 8.5-9.5, and the reaction is performed at 20-35° C. with shaking in a dark for 1-3 h or at 4° C. overnight, and specifically, the reaction is performed at pH 7.5 and 25° C. with shaking in a dark for 2 h. High-performance liquid chromatography is preferably employed for separating and purifying a precursor compound, and a product at a corresponding separation peak is collected to obtain a precursor compound.

In S2, the metallic radionuclide eluent varies slightly, depending on the radionuclide selected. For example, a metallic radionuclide ⁶⁸Ga eluent is a hydrochloric acid solution containing ⁶⁸GaCl₃. Specifically, a mixed solution of radioactive ⁶⁸GaCl₃ and hydrochloric acid is obtained through a reaction that a germanium-gallium generator is eluted with hydrochloric acid. A concentration of HCl for elution is adjusted as needed, which is optionally 0.01-0.1 M, and specifically 0.05 M. After the metallic radionuclide eluent is obtained, an acidic environment is created through pH adjustment (pH 4.0-4.5) with a buffer solution, and then the precursor compound is added to form a chelation reaction system.

Preparation methods for different metallic radionuclide eluents are conventional techniques in the art, which are not described in detail herein. Additionally, it is to be noted that reaction mechanisms of different metallic radionuclide eluents and the precursor compound of the present disclosure are basically the same, both of which rely on coordination bonding between metal and the bifunctional chelator. Therefore, different eluents basically exert negligible impact on chelation reactions.

Optionally, in S2, a dosage ratio of the metallic radionuclide to the precursor compound is 30-50 MBq:1 nM. In other words, the metallic radionuclide is added based on an activity, and the metallic radionuclide with an activity of 30-50 MBq is added per 1 nM of the precursor compound, where the activity is preferably 37 MBq.

Optionally, in S2, the buffer solution is a sodium acetate buffer solution, with a concentration of 0.1-1M.

Optionally, in S2, during a chelation reaction, a pH value of the reaction is 4.0-4.5, the reaction is performed at 90-100° C. with shaking for 10-20 min, and specifically, the reaction is performed at pH 4.0 and 95° C. with shaking for 15 min. A solid-phase extraction C18 column is preferably used to separate and purify the myocarditis-targeted radionuclide molecular probe, and a structure of a synthesized product is further verified through mass spectrometry and ultraviolet spectroscopy.

In another example, the present disclosure provides an application of the above myocarditis-targeted radionuclide molecular probe in preparing a myocarditis imaging agent, and particularly an application thereof in preparing a viral myocarditis imaging agent.

The application of the myocarditis-targeted radionuclide molecular probe in preparing a myocarditis imaging agent, and the above myocarditis-targeted radionuclide molecular probe have the same advantages over the prior art, which are not described in detail herein.

Based on the same inventive concept as above, a myocarditis imaging agent is provided in another example of the present disclosure, including the above myocarditis-targeted radionuclide molecular probe.

The myocarditis imaging agent and the above myocarditis-targeted radionuclide molecular probe have the same advantages over the prior art, which are not described in detail herein.

The present disclosure is further described below with reference to specific examples. If specific conditions of experimental methods are not specified in the following examples, conditions recommended by the manufacturer shall prevail.

In the following examples, the DPP4 inhibitor is Linagliptin, the metallic radionuclide is ⁶⁸Ga, the bifunctional chelator is p-SCN-Bn-DOTA (purchased from Macrocyclics America, Catalog No. B-205), and a synthesis route for ⁶⁸Ga-p-SCN-Bn-DOTA-Linagliptin radionuclide molecular probes is as follows:

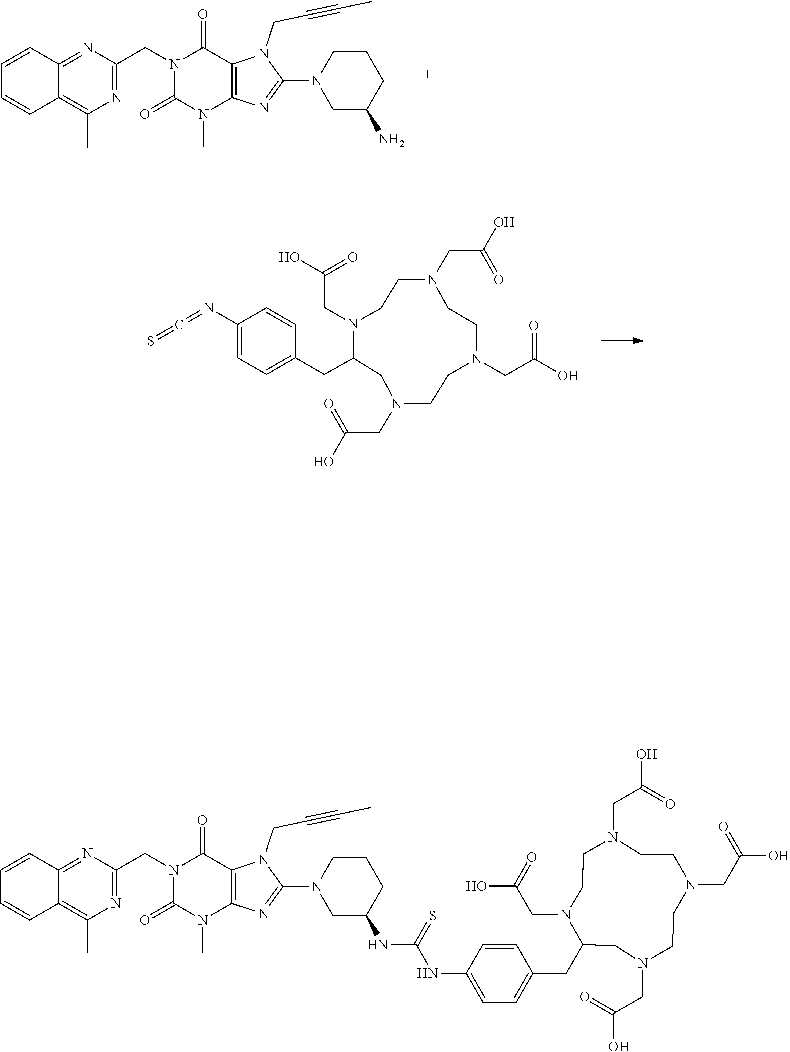 [SMILES: CC#CCn1c(N2CCC[C@@H](N)C2)nc2c1c(=O)n(Cc1nc(C)c3ccccc3n1)c(=O)n2C ; O=C(O)CN1CCN(CC(=O)O)CCN(CC(=O)O)C(Cc2ccc(N=C=S)cc2)CN(CC(=O)O)CC1 ; CC#CCn1c(N2CCC[C@@H](NC(=S)Nc3ccc(CC4CN(CC(=O)O)CCN(CC(=O)O)CCN(CC(=O)O)CCN4CC(=O)O)cc3)C2)nc2c1c(=O)n(Cc1nc(C)c3ccccc3n1)c(=O)n2C]

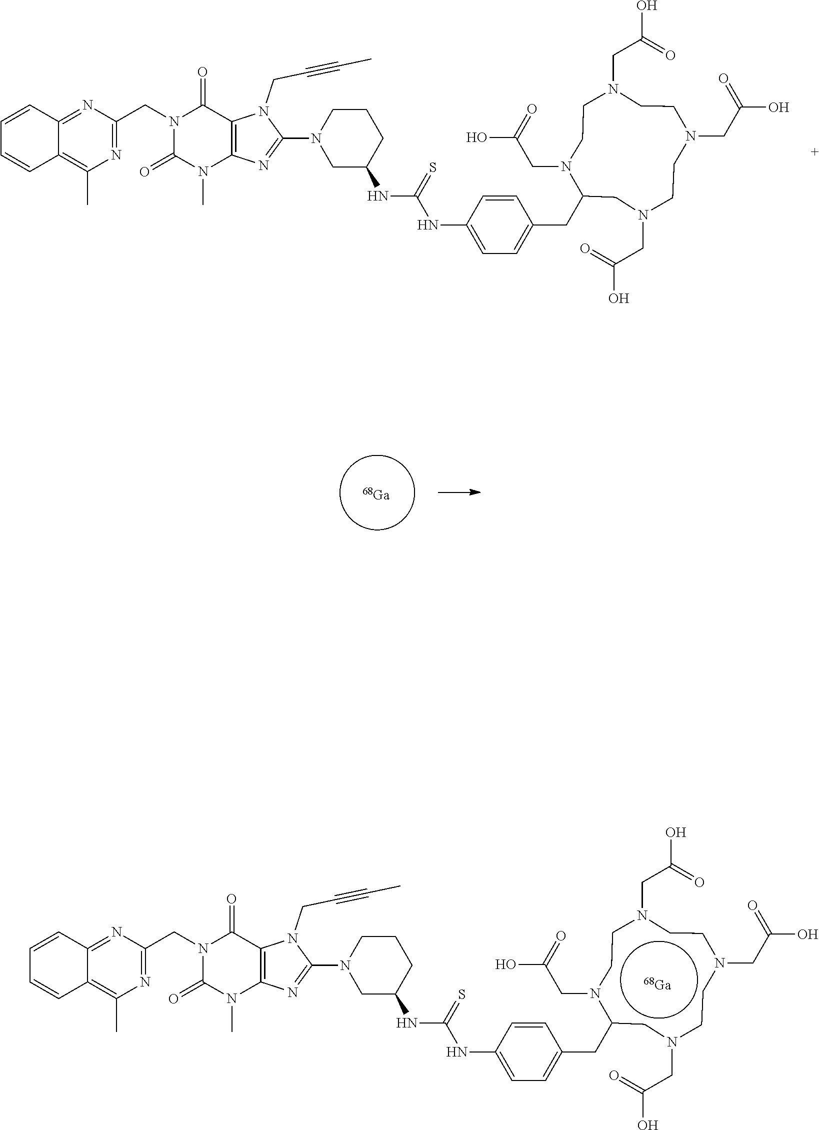

Reaction processes of DPP4 inhibitors such as Sitagliptin, Saxagliptin, Alogliptin and Gemigliptin and bifunctional chelators such as p-SCN-Bn-NOTA, p-SCN-Bn-DOTA, p-SCN-Bn-PCTA, p-SCN-Bn-HEHA, p-SCN-Bn-TCMC, and p-SCN-Bn-DTPA are the same as above.

### Example 1 Myocarditis-Targeted Radionuclide Molecular Probe and Preparation Method Thereof

A myocarditis-targeted radionuclide molecular probe, includes Linagliptin, a bifunctional chelator p-SCN-Bn-DOTA covalently bonded to an amino group of the Linagliptin, and a metallic radionuclide ⁶⁸Ga coordinately bonded to the bifunctional chelator, with a structural formula thereof as follows:

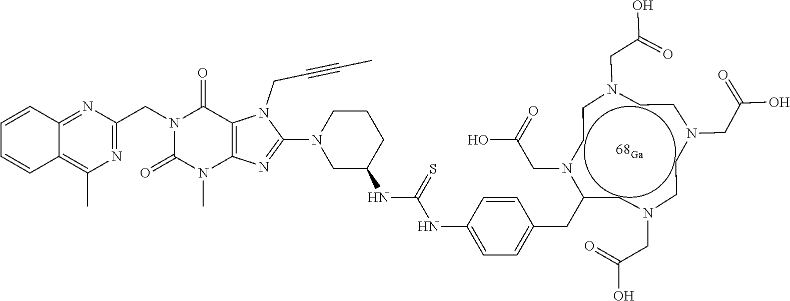

The preparation method therefor includes the following steps:
S1, Linagliptin and a bifunctional chelator p-SCN-Bn-DOTA were dissolved in an organic solvent DMSO at a molar ratio of 1.2:1, a pH value of reaction was adjusted to 7.5, the reaction was performed at 25° C. with shaking in a dark for 2 h, and separation and purification were performed through high-performance liquid chromatography after the reaction to obtain a precursor compound.

Steps for the separation and purification through high-performance liquid chromatography are as follows: C18 light Sep-Pak cartridge (Waters, USA) was used as a chromatographic column and 15-35% acetonitrile as a mobile phase, linear gradient elution was performed for 0-20 min, an acetonitrile concentration was increased from 15% to 35% at a flow rate of 1 mL/min, and then elution was performed for 30 min at the above concentration; and an eluent was collected for 26-28 min, and dried with nitrogen to obtain a precursor compound.

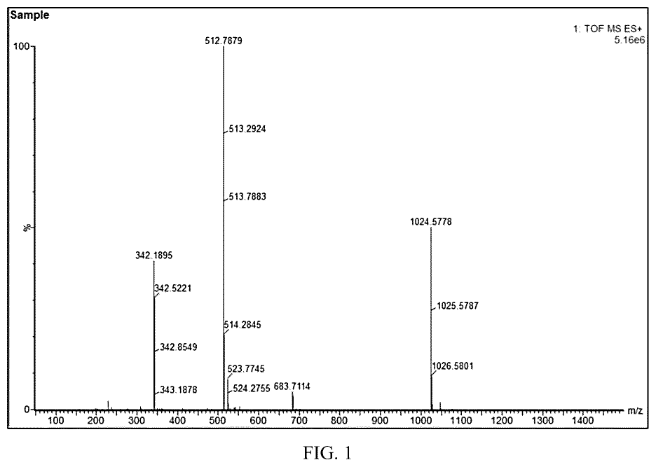

The liquid chromatography-mass spectrometry (LC-MS) of the precursor compound is shown in FIG. 1. It is seen that a molecular weight of a target product was 1023.4385. When the target product carried one charge, the molecular weight displayed by LC-MS was 1024.44, when the target product carried two charges, the molecular weight displayed by LC-MS was 512.73, when the target product carried three charges, the molecular weight displayed by LC-MS was 342.15, and when the target product carried four charges, the molecular weight displayed by LC-MS was 256.87. The mass spectrogram showed that the molecular weight of the target product was 1023.44, with slight variations in charge, indicating that the target product was successfully synthesized.

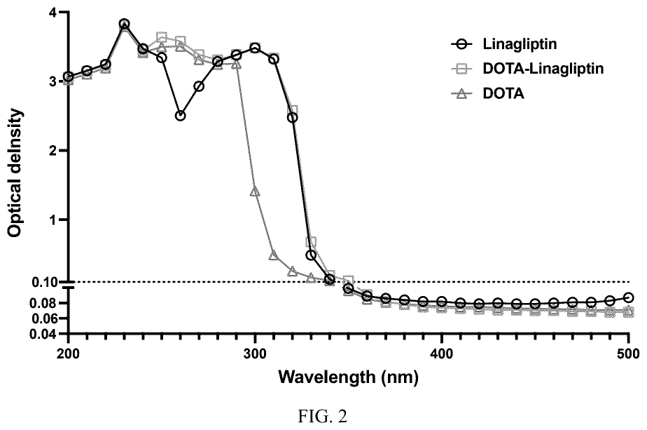

The ultraviolet spectrogram of the precursor compound is shown in FIG. 2, where DOTA represents p-SCN-Bn-DOTA, HY19285 represents Linagliptin. It is seen from the figure that the purified product DOTA-Linagliptin contains characteristic UV absorption peaks of DOTA and Linagliptin.

S2, a hydrochloric acid solution containing ⁶⁸GaCl₃ as an eluent containing ⁶⁸GaCl₃ was obtained through a reaction that a germanium-gallium generator was eluted with hydrochloric acid, a pH value was adjusted to 4.0 by using a sodium acetate buffer solution with a concentration of 0.25 M, the purified precursor compound was added and mixed well to obtain a reaction mixture containing ⁶⁸GaCl₃ with an activity of 111 MBq and 3 nM of the precursor compound, the reaction was performed at 95° C. with shaking for 15 min, and the mixture was separated and purified with a solid-phase extraction C18 column after the reaction to obtain a myocarditis-targeted radionuclide molecular probe.

The mixture was separated and purified with a solid-phase extraction C18 column as follows: 1) activate the C18 column: 10 mL of anhydrous ethanol and 10 mL of ultrapure water were sequentially added dropwise; 2) purify the C18 column: a reaction solution was loaded onto the C18 column, after dropwise addition of a mixed reaction solution, 10 mL of ultrapure water was added dropwise to remove free ⁶⁸Ga, and then 0.5 mL of anhydrous ethanol was quickly pushed through to obtain a radionuclide molecular probe of a product ⁶⁸Ga-p-SCN-Bn-DOTA-Linagliptin radionuclide, which is hereinafter referred to as ⁶⁸Ga-DOTA-Linagliptin.

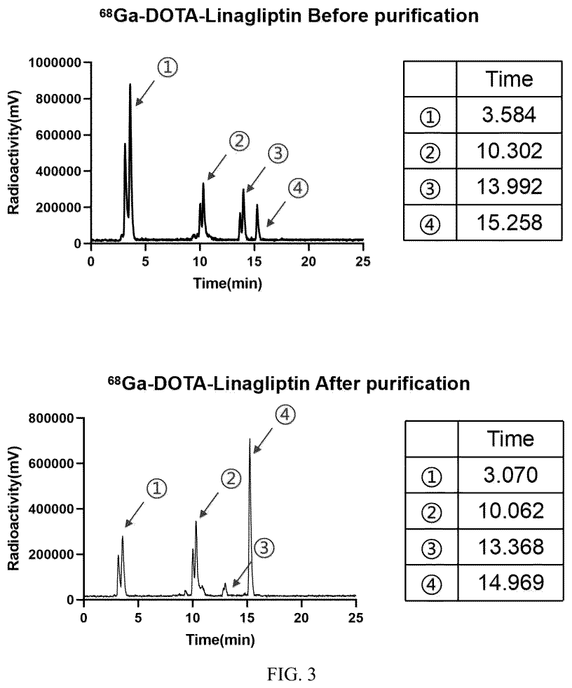

High-performance liquid chromatograms of the ⁶⁸Ga-DOTA-Linagliptin probe before and after purification are shown in FIG. 3, where peak \textcircled{1} represents free ⁶⁸Ga, peak \textcircled{4} represents ⁶⁸Ga-DOTA-Linagliptin, and peaks \textcircled{2} and \textcircled{3} represent impurities. It is seen from the figure that after purification of the C18 column, most of obtained products are ⁶⁸Ga-DOTA-Linagliptin.

### Example 2 Analysis of DPP4 Protein Expression in Cardiac Tissues of Mice with Myocarditis

Modeling of mice with myocarditis: The coxsackievirus B3 (CVB3) used for viral myocarditis modeling was sourced from Wuhan Institute of Virology, CAS, and was amplified and passaged in Hela cells. With reference to relevant literature “Chen Liang. Study on inhibition of CVB3-induced viral myocarditis by CYP2J2/EETs and its mechanism [D]. Huazhong University of Science and Technology, 2018.”, 4-week-old male BALB/c mice (sourced from Jiangsu GemPharmatech Co., Ltd.) were selected and intraperitoneally injected with 150 μL of virus solution at a dose of 10⁵ TCID50 to establish a model. Mice in a control group were intraperitoneally injected with 150 μL of a PBS solution.

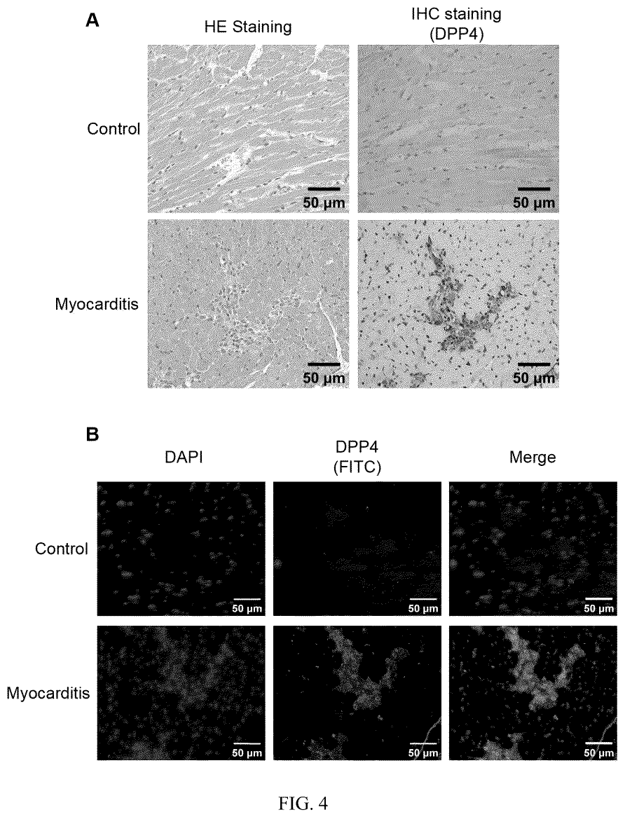

Cardiac tissues of model mice were collected on the seventh dayay after CVB3 virus infection, and paraffin sections were prepared and subjected to HE staining, immunohistochemical staining and immunofluorescence analysis respectively (a DPP4 antibody was purchased from RD Company, Catalog No. AF954), with results shown in FIG. 4.

Seen from the paraffin sections of hearts of mice in the control group as shown in FIG. 4, inflammatory cell infiltration was not observed in myocardial tissues, and results of immunohistochemical staining and immunofluorescence analysis showed an extremely low expression of DPP4. Seen from the paraffin sections of cardiac tissues of model mice collected on the seventh dayay after CVB3 virus infection, extensive inflammatory cell infiltration (indicated by solid black arrows) was observed, and results of immunohistochemical staining and immunofluorescence analysis showed a significant increase in the expression of DPP4 protein. The above findings demonstrate that the DPP4 protein is specifically and highly expressed in inflammatory cell infiltration regions of viral myocarditis, and cardiomyocytes and cardiac fibroblasts in non-inflammatory regions hardly express the DPP4 protein. Therefore, DPP4 serves as a promising specific target for myocarditis imaging applications.

### Example 3 Affinity Testing of Myocarditis-Targeted Radionuclide Molecular Probes with 293T Cells Overexpressing DPP4 Protein

Ordinary 293T cells (purchased from ATCC) with an extremely low expression of DPP4 protein were used for experimental control (293T-Control). Overexpression of DPP4 by lentiviral transfection enabled to obtain a 293T cell line with high DPP4 protein expression (293T-DPP4 cells), where a DPP4 transcript sequence ID was NM001935.4, a protein sequence ID was NP001926.2, and a coding sequence was 2301 bp.

The targeting capability and specificity of the radionuclide molecular probe were verified through in vitro cellular uptake; and 293T cells and 293T-DPP4 cells for the control group were conventionally cultured in vitro, the myocarditis-targeted radionuclide molecular probe synthesized in Example 1 was added to a culture medium, and after incubation for appropriate time, radioactive cellular uptake values were detected to evaluate binding affinity of the probe with the DPP4 protein in vitro. The specific steps are as follows: frozen 293T-Control and 293T-DPP4 cell lines were thawn, routinely cultured and expanded, the two cell lines were digested and resuspended with good morphology, and then inoculated in a 24-well plate, and when the cells were attached with a density of about 70-80%, a supernatant was discarded, and starving was performed for 2 h with a serum-free high-glucose DMEM medium. A radionuclide molecular probe was dissolved in the serum-free high-glucose DMEM medium, 1 mL of serum-free culture medium containing 2 μCi of radionuclide molecular probe was added to each well of the 24-well plate, a supernatant and a cell layer lysed by NaOH were collected at time points of 30 min and 60 min respectively, radioactive counts of the supernatant and the cell layer were determined by using a γ-counter, and an uptake rate was calculated.

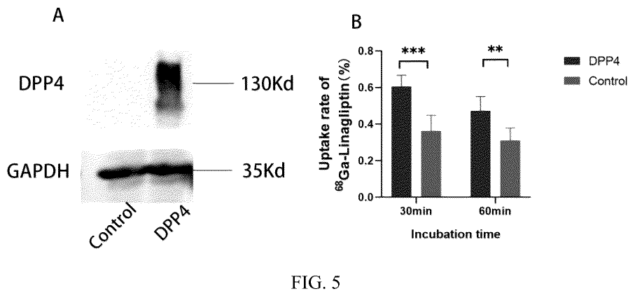

FIG. 5 shows experimental results of cellular uptake with a radionuclide molecular probe ⁶⁸Ga-DOTA-Linagliptin according to the present disclosure, where FIG. 5A shows expression levels of DPP4 protein in 293T cells overexpressing DPP4. A Western Blot method was used for detecting, a DPP4 antibody was purchased from RD Company, Catalog No. AF954, and a GAPDH protein was used as an internal reference (purchased from Proteintech Company, Catalog No. 60004-1). FIG. 5B shows uptake rates of 293T cells highly expressing DPP4, where Control represents 293T cells of mice in the control group, and DPP4 represents 293T cells overexpressing DPP4. It is seen from the figure that 293T cells (293T-Control) had an extremely low expression of DPP4 protein, a 293T cell line with high DPP4 protein expression (293T-DPP4 cells) was obtained through transfection and overexpression of DPP4, and compared with the 293T cells in the control group, the 293T-DPP4 cells exhibited a significantly higher uptake rate of the radionuclide molecular probe, indicating that the probe had higher in vitro binding affinity.

Moreover, each mouse in a DPP4 group was subcutaneously injected with 100 μL of suspension containing 4.5×10⁶ 293T-DPP4 cells in an axillary region thereof, and the control group did not receive any treatment. Two hours after injection, the mice were imaged with the radionuclide molecular probe to determine in vivo affinity of the probe for DPP4-293T cells.

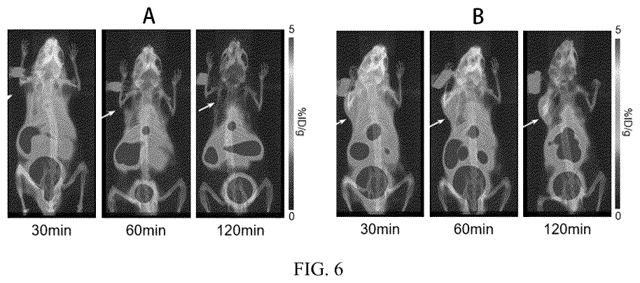

FIG. 6 illustrates experimental results of in vivo targeting of a radionuclide molecular probe ⁶⁸Ga-DOTA-Linagliptin according to the present disclosure, where FIG. 6A shows imaging results of mice not subcutaneously injected at axillary regions thereof, and FIG. 6B shows imaging results of mice subcutaneously injected with a suspension of 293T-DPP4 cells at axillary regions thereof. As shown in the figure, corresponding preparations were injected subcutaneously at axillary regions thereof indicated by white arrows, and then the radionuclide molecular probe was used for imaging. It is seen that obvious radiotracer accumulation was observed at 293T-DPP4 cell injection sites, while no obvious accumulation was observed at axillary regions of mice in the control group.

### Example 4: Imaging Effects and Biodistribution of Myocarditis-Targeted Radionuclide Molecular Probe in Myocarditis Model Mice

A modeling method for mice with viral myocarditis is the same as that in Example 2. PET/CT imaging monitoring was performed for a same mouse on the third day before virus infection and the seventh day after virus infection by using a radionuclide molecular probe, and 30 min later after injection of radionuclide molecular probe into other mice treated during the same period, main organs were collected to detect quantitative biodistribution in a γ-counter.

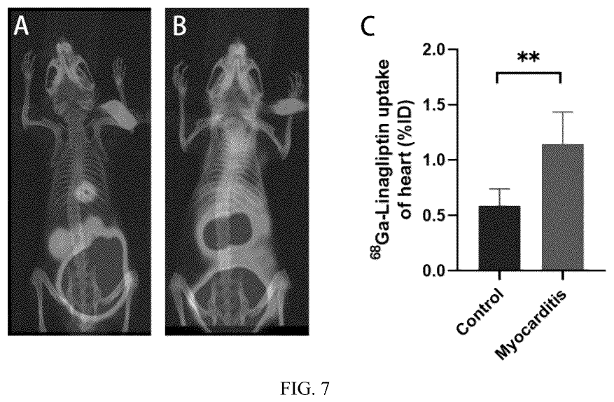

FIG. 7 illustrates tracking imaging results of the same mouse before and after modeling with a radionuclide molecular probe ⁶⁸Ga-DOTA-Linagliptin, where FIG. 7A shows imaging results of the mouse (from the Control group) on the third day before viral infection, FIG. 7B shows imaging results of the mouse (from the Myocarditis group) on the seventh day after viral infection, and FIG. C is a statistical graph showing imaging results on radiation uptake rates of cardiac region of the mouse before and after viral infection. The results showed that radiation uptake values of cardiac regions of the mice before infection with CVB3 were low, and when extensive inflammatory infiltration of hearts was observed on the seventh day after virus infection, the radiation uptake values of cardiac regions of the mice increased significantly.

Table 1 shows biodistribution of radionuclide molecular probes in % ID/g, and data are expressed by mean±standard deviation. Quantitative results of the biodistribution of radionuclide molecular probes are basically consistent with imaging results. It is seen that the probe has high affinity for myocardial inflammatory foci highly expressing DPP4. Cardiac uptake values of mice in the myocarditis group are 0.94±0.15, and cardiac uptake values of mice in the control group are only 0.41±0.21 (p<0.05), demonstrating specific high uptake and low uptake in non-target tissues. A radioactivity concentration is significantly different from that of adjacent tissues, and the radionuclide molecular probe with high detection sensitivity is used to achieve early diagnosis and efficacy evaluation of myocarditis.

TABLE 1
In vivo biodistribution of the radionuclide molecular probe
of the present disclosure in myocarditis model mice

| | Control group | Myocarditis group | |
|---|---|---|---|
| Tissue | (n = 3) | (n = 3) | p-value |
| Blood | 1.12 ± 0.71 | 1.68 ± 0.26 | 0.267 |
| Brain | 0.09 ± 0.03 | 0.13 ± 0.01 | 0.066 |
| Heart | 0.41 ± 0.21 | 0.94 ± 0.15 | 0.025 |
| Lung | 0.4 ± 0.26 | 1.4 ± 0.36 | 0.017 |
| Liver | 0.45 ± 0.16 | 1.27 ± 0.22 | 0.006 |
| Spleen | 0.36 ± 0.03 | 0.77 ± 0.09 | 0.002 |
| Kidney | 2.25 ± 0.92 | 6.57 ± 2.51 | 0.049 |
| Stomach | 0.32 ± 0.18 | 1.13 ± 0.25 | 0.01 |
| Small intestine | 0.49 ± 0.33 | 0.82 ± 0.18 | 0.199 |
| Large intestine | 0.52 ± 0.25 | 0.85 ± 0.41 | 0.31 |
| Muscle | 2.88 ± 2.1 | 1.13 ± 0.29 | 0.226 |
| Bone | 1.6 ± 0.33 | 1.56 ± 0.25 | 0.878 |
| Gallbladder | 5.16 ± 1.51 | 2.2 ± 0.91 | 0.066 |

The above are merely preferred embodiments of the present disclosure, and are not intended to limit the present disclosure. Any modifications, equivalent replacements and improvements made within the spirit and principle of the present disclosure should fall within the protection scope of the present disclosure.

What is claimed is:

1. A myocarditis-targeted radionuclide molecular probe, comprising a DPP4 inhibitor, a bifunctional chelator covalently bonded to an amino group of the DPP4 inhibitor, and a metallic radionuclide coordinately bonded to the bifunctional chelator, wherein the DPP4 inhibitor is Linagliptin, the bifunctional chelator is p-SCN-Bn-DOTA, and the metallic radionuclide is ⁶⁸Ga.

2. A preparation method for a myocarditis-targeted radionuclide molecular probe, used for preparing the myocarditis-targeted radionuclide molecular probe according to claim 1, comprising the following steps:
S1, dissolving a DPP4 inhibitor and a bifunctional chelator in an organic solvent, reacting same under alkaline conditions, and purifying after the reaction to obtain a precursor compound; and
S2, adding a metallic radionuclide eluent and the precursor compound into a buffer solution to perform a metallic radionuclide labeling reaction, and separating and purifying after the reaction to obtain a myocarditis-targeted radionuclide molecular probe.

3. The preparation method for a myocarditis-targeted radionuclide molecular probe according to claim 2, wherein in S1, a molar ratio of the DPP4 inhibitor to the bifunctional chelator is 1-2:1;
the organic solvent is dimethyl sulfoxide, methanol or chloroform; and
a pH value of reaction is 8.5-9.5, and the reaction is performed at 20-35° C. with shaking in a dark for 1-3 h or at 4° C. overnight.

4. The preparation method for a myocarditis-targeted radionuclide molecular probe according to claim 2, wherein in S2, a dosage ratio of metallic radionuclide in the metallic radionuclide eluent to the precursor compound is 30-50 MBq:1 nM;
the buffer solution is a sodium acetate buffer solution with a concentration of 0.1-1 M; and
a pH value of the reaction is 4.0-4.5, and the reaction is performed at 90-100° C. with shaking for 10-20 min.

5. An application of the myocarditis-targeted radionuclide molecular probe according to claim 1 in preparing a myocarditis imaging agent.

6. A myocarditis imaging agent, comprising the myocarditis-targeted radionuclide molecular probe according to claim 1.
